(19) United States
(12) Patent Application Publication
(10) Pub. No.: US 2016/0143542 A1
(43) Pub. Date: May 26, 2016

(54) Minimally Invasive Optical Photoacoustic Endoscopy with a Single Waveguide for Light and Sound

(71) Applicant: Ecole Polytechnique Fédérale de Lausanne (EPFL), Lausanne (CH)

(72) Inventors: Emmanuel Bossy, Neuilly-Plaisance (FR); Jean-Pierre Huignard, Paris (FR); Olivier Simandoux, Paris (FR); Christophe Moser, Lausanne (CH); Demetri Psaltis, Preverenges (CH); Ioannis Papadopoulos, Lausanne (CH); Nicolino Stasio, Lausanne (CH); Salma Farahi, Lausanne (CH)

(21) Appl. No.: 14/948,704

(22) Filed: Nov. 23, 2015

(30) Foreign Application Priority Data
Nov. 24, 2014 (IB) PCT/IB2014/066289

(51) Int. Cl. A61B 5/00; A61B 1/06; A61B 1/07; A61B 1/00

(52) U.S. Cl. CPC A61B 5/0095; A61B 1/00009; A61B 1/063; A61B 1/0661; A61B 1/0653; A61B 1/07

(57) ABSTRACT

An endoscopic device for photoacoustic imaging, including a multimode optical fiber having a distal end and a proximal end, a light source to provide a light beam to the proximal end of the multimode optical fiber, a transducer to capture acoustic waves that are emitted from the proximal end of the multimode optical fiber, and a processing device to generate a photoacoustic image based on data from the captured acoustic waves captured by the transducer, wherein the distal end of the multimode optical fiber is configured to be inserted into a sample, the sample generating the acoustic waves by a photoacoustic effect.

### CROSS REFERENCE TO RELATED APPLICATIONS

This application claims priority to the International Application PCT/IB2014/066289, with an international filing date of Nov. 24, 2014, the entire contents thereof are herewith incorporated by reference.

### FIELD OF THE INVENTION

This invention relates to a method, system, and device for minimally invasive optical-resolution photoacoustic endoscopy. Particularly, it relates to using a multimode waveguide having optical-in and acoustic-out properties, to perform both optical excitation and remote acoustic detection at the same tip of the waveguide, in the context of photoacoustic endoscopy, for example photoacoustic imaging at depth into a sample such as biological tissue.

### DISCUSSION OF THE BACKGROUND ART

Photoacoustic microscopy (PAM) is a rapidly evolving imaging technique that is capable of delivering multi-scale images based on the optical absorption properties of the investigated sample. See Paul Beard. “Biomedical photoacoustic imaging,” Interface Focus, Vol. 1, No. 4, pp. 602-631, 2011. Absorption of laser pulses by the sample locally generates acoustic waves via the photoacoustic effect. An acoustic transducer is generally used to detect the generated acoustic waves enabling the formation of an image. One of the advantages of photoacoustic microscopy is that it can provide either label-free images of biological tissues, in which case the contrast rises from the intrinsic variation of optical absorption coefficient. See H. F. Zhang, K. Maslov, G. Stoica and L. V. Wang. “Functional photoacoustic microscopy for high-resolution and noninvasive in vivo imaging,” Nature Biotechnology, Vol. 24, No. 7, pp. 848-851, 2006. See also K. Maslov, H. F. Zhang, S. Hu and L. V. Wang. “Optical-resolution photoacoustic microscopy for in vivo imaging of single capillaries,” Optics Letters, Vol. 33, No. 9, pp. 929-931, 2008. Also, images of tissue based on exogenous contrast agents can be used to target and image specific structures and metabolisms. Photoacoustic microscopy approaches can be divided in two categories, based on the image resolution. In acoustic-resolution photoacoustic microscopy (AR-PAM), the image resolution is dictated by the frequency response of the ultrasound detection device. With this approach, images with resolution ranging from millimeter to less than a hundred microns can be obtained at depths much larger than the optical transport mean free path, typically 1 mm in biological tissue, where purely optical techniques are limited by multiple scattering. See V. Ntziachristos. “Going deeper than microscopy: the optical imaging frontier in biology,” Nature Methods, Vol. 7, No. 8, pp. 603-614, 2010. The image reconstruction is based on acquiring ultrasound signals for different positions of the transducers, either by scanning a single element transducer or by using multi-element probes.

In optical-resolution photoacoustic microscopy, the excitation light is focused into a diffraction-limited spot, usually with an objective lens. In this configuration the optical energy is deposited on the sample, and therefore the region that generates the acoustic waves, are limited only to the illuminated diffraction-limited voxel, yielding photoacoustic images with optical resolution. See Z. Xie, S. Jiao, H. F. Zhang and C. A. Puliafito. “Laser-scanning optical-resolution photoacoustic microscopy,” Optics Letters, Vol. 34, No. 12, pp. 1771-1773, 2009, see also P. Hajireza, W. Shi and R. J. Zemp. “Label-free in vivo fiber-based optical-resolution photoacoustic microscopy,” Optics Letters, Vol. 36, No. 20, pp. 4107-4109, 2011. The generation of full-sized images is based on the raster scanning of the optical excitation spot. However, as OR-PAM relies on the ability to focus light into the sample of interest, it is feasible only within the ballistic regime of optical propagation in scattering media, therefore generally limited to penetration depths smaller than 1 mm for biological tissue.

While the imaging depth of AR-PAM is limited by the so called hard limit of optical penetration, dominated by the absorption properties of the investigated tissue, the imaging depth of optical-resolution photoacoustic microscopy (OR-PAM) is limited by the optical transport mean free path. In order to overcome these limitations, endoscopic modalities have been deployed for the acquisition of photoacoustic images deep inside tissue cavities. Similar to conventional optical endoscopy, fiber bundle endoscopes have been adapted to add photoacoustic imaging capabilities. See P. Shao, W. Shi, P. Hajireza and R. J. Zemp. “Integrated micro-endoscopy system for simultaneous fluorescence and optical-resolution photoacoustic imaging,” Journal of Biomedical Optics, Vol. 17, No. 7, 2012. A different approach of a purely photoacoustic endoscope has also been proposed. See J.-M. Yang, K. Maslov, H.-C. Yang, Q. Zhou, K. K. Shung and L. V. Wang. “Photoacoustic endoscopy,” Optics Letters, Vol. 34, No. 10, pp. 1591-1593, 2009, see also Y. Yuan, S. Yang and D. Xing. “Preclinical photoacoustic imaging endoscope based on acousto-optic coaxial system using ring transducer array,” Optics Letters, Vol. 35, No. 13, pp. 2266-2268, 2010. In these publications, a single mode fiber is used for the delivery of a diffused excitation optical field and the integrated transducer picks up the acoustic signal. These devices are equipped with a rotational motor so that the excitation and detected field can be scanned around the endoscope's axis, and these devices are still limited in terms of resolution, ˜58 μm lateral resolution and the smallest achieved diameter of the endoscopic head was 2.5 mm. See J.-M. Yang, R. Chen, C. Favazza, J. Yao, C. Li, Z. Hu, Q. Zhou, K. K. Shung and L. V. Wang. “A 2.5-mm diameter probe for photoacoustic and ultrasonic endoscopy,” Optics Express, Vol. 20, No. 21, pp. 23944-23953, 2012.

Fiber bundle endoscopes, where each of the single mode fibers of the device acts as a single pixel of the final image, dominate the commercial domain, however the resolution is limited by the distance between adjacent fiber cores, usually ˜5 μm. See A. F. Gmitro and D. Aziz. “Confocal microscopy through a fiberoptic imaging bundle,” Optics Letters, Vol. 18, No. 8, pp. 565-567, 1993. Designs that combine optical fibers, conventional focusing optical elements and mechanical actuators have been used in versatile high resolution endoscopes. See D. Bird and M. Gu. “Two-photon fluorescence endoscopy with a micro-optic scanning head,” Optics Letters, Vol. 28, No. 17, pp. 1552-1554, 2003. Yet these designs remain relatively large, i.e. larger than 2 mm.

Recently, the possibility of building functional ultra-thin imaging devices has been explored, based solely on multimode optical fibers using the large number of degrees of freedom available in these waveguides. See S. Bianchi and R. Di Leonardo. “A multi-mode fiber probe for holographic micromanipulation and microscopy,” Lab on a Chip, Vol. 12, No. 3, pp. 635-639, 2012, see also Y. Choi, C. Yoon, M. Kim, T. D. Yang, C. Fang-Yen, R. R. Dasari, K. J. Lee and W. Choi. “Scanner-Free and Wide-Field Endoscopic Imaging by Using a Single Multimode Optical Fiber,” Physical Review Letters, Vol. 109, No. 20, 2012, see also T. Cizmar and K. Dholakia. “Exploiting multimode waveguides for pure fibre-based imaging,” Nature Communications, Vol. 3, 2012, see also I. N. Papadopoulos, S. Farahi, C. Moser and D. Psaltis. “Focusing and scanning light through a multimode optical fiber using digital phase conjugation,” Optics Express, Vol. 20, No. 10, pp. 10583-10590, 2012, see also R. N. Mahalati, R. Y. Gu and J. M. Kahn. “Resolution limits for imaging through multi-mode fiber,” Optics Express, Vol. 21, No. 2, pp. 1656-1668, 2013, see also I. N. Papadopoulos, S. Farahi, C. Moser and D. Psaltis. “High-resolution, lensless endoscope based on digital scanning through a multimode optical fiber,” Biomedical Optics Express, Vol. 4, No. 2, pp. 260-270, 2013.

In particular, researchers have recently proposed and demonstrated an optical-resolution photoacoustic imaging modality in which a multimode optical fiber is used as the source of the optical excitation field. See I. N. Papadopoulos, O. Simandoux, S. Farahi, J. P. Huignard, E. Bossy, D. Psaltis and C. Moser. “Optical-resolution photoacoustic microscopy by use of a multimode fiber,” Applied Physics Letters, Vol. 102, No. 21, 2013. Digital phase conjugation was used to focus and digitally scan a diffraction-limited spot at the distal tip of the multimode fiber, therefore eliminating the need for optical lenses and mechanical actuators. The small diameters of multimode fibers along with their ability to focus light into a micron-sized spot, pave the way for the implementation of optical resolution PAM, deeper than the ballistic regime of light propagation. In the configuration illustrated on FIG. 1, a needle-type endoscope can be used for the optical excitation by directly penetrating the tissue and bringing the optical excitation directly against the sample of interest, and FIG. 2 illustrates the results obtained with this approach.

The above discussed approaches appear to address the problem of optical-focusing through a small diameter device inserted into tissue, ultrasound needs to be detected after is has been generated via the photoacoustic effect. Because OR-PAM generates photoacoustic waves with frequencies of typically several tens of MHz, the ability to detect externally the high frequency photoacoustic waves generated at depth in tissue is limited by the acoustic attenuation, typically 0.1-1.0 dB/cm/MHz. Also, OR-PAM endoscopy approaches have been introduced recently. See P. Hajireza, W. Shi and R. Zemp. “Label-free in vivo GRIN-lens optical resolution photoacoustic micro-endoscopy,” Laser Physics Letters, Vol. 10, No. 5, 2013. In this publication, ultrasound is detected from outside the sample either through a relatively thin tissue thickness in the MHz range, or through a non-absorbing tissue-mimicking samples. Internal detection can be considered, but ultrasound sensors have to be miniaturized, resulting in limited sensitivity, and requiring dedicated technological developments. See E. Z. Zhang and P. C. Beard. “A miniature all-optical photoacoustic imaging probe,” Photons Plus Ultrasound: Imaging and Sensing 2011, p. 7899, 2011.

Despite all these advancements in photoacoustic endoscopy, no endoscopic device is currently able to achieve both optical scanning and focusing and detection of the generated photoacoustic waves to generate an OR-PAM image at centimeters depth in tissue. Therefore, novel technologies and principles in photoacoustic microscopy are desired.

### SUMMARY

According to one aspect of the present invention, an endoscopic device for photoacoustic imaging is provided. The endoscopic device preferably includes a multimode waveguide having a distal end and a proximal end, a light source to provide a light beam to the proximal end of the multimode waveguide, and a transducer to capture acoustic radiation that is emitted from the proximal end of the multimode waveguide. Moreover, the endoscopic device preferably further includes a processing device to generate a photoacoustic image based on data from the captured acoustic radiation captured by the transducer, and the distal end of the multimode waveguide is configured to be inserted into a sample, the sample generating the acoustic radiation by a photoacoustic effect.

According to another aspect of the present invention, a method to generate a photoacoustic image from a sample with a multimode waveguide is provided. Preferably, the multimode waveguide penetrates into the sample such that a distal end of the multimode waveguide faces an area of the sample under test inside the sample. In addition, preferably the method includes the steps of radiating a proximal end of the multimode waveguide with light from a light source, and guiding the light through the multimode waveguide and guiding sound through the multimode waveguide, the sound being created by the light that exits the distal end of the multimode waveguide and impinges on the area of the sample under test, the area causing a photoacoustic effect generating acoustic radiation that enters the multimode waveguide by the distal end. Moreover, the method also preferably includes the step of emitting the sound from the proximal end of the multimode waveguide, and capturing the emitted sound by a transducer to generate the photoacoustic image.

According to yet another aspect of the present invention, an endoscopic system for photoacoustic imaging is provided. The system preferably includes a sample having an opening, a dual waveguide having a distal end and a proximal end, the distal end of the dual waveguide arranged inside the opening, an area of the sample facing the distal end of the dual wave guide being under test, and a light source to provide a light beam to the proximal end of the dual waveguide. Moreover, the system preferably includes a transducer to capture acoustic radiation that is emitted from the proximal end of the dual waveguide, and a processing device to generate a photoacoustic image based on data from the captured acoustic radiation captured by the transducer, and the acoustic radiation is generated by a photoacoustic effect at the area of the sample, by the acoustic radiation that enters the dual waveguide at the distal end.

The above and other objects, features and advantages of the present invention and the manner of realizing them will become more apparent, and the invention itself will best be understood from a study of the following description and appended claims with reference to the attached drawings showing some preferred embodiments of the invention.

### BRIEF DESCRIPTION OF THE SEVERAL VIEWS OF THE DRAWINGS

The accompanying drawings, which are incorporated herein and constitute part of this specification, illustrate the presently preferred embodiments of the invention, and together with the general description given above and the detailed description given below, serve to explain features of the invention.

FIG. 1 depicts a schematic representation of the experimental setup for the generation of optical resolution photoacoustic microscopy images using a multimode fiber for the optical excitation according to the background art, where the multi-mode fiber was used to guide light to the sample on the distal tip, and the ultrasound detection was performed after propagation from the sample at the distal tip through water to the transducer;

FIG. 2 depicts on the left side a photoacoustic image of a knot obtained after light was propagated and focused through a multimode fiber, and on the right side a white light photograph of the sample according to the background art;

FIG. 3 shows a schematic representation of sound guiding by the water-filled core of a silica capillary of the waveguide according to one aspect of the present invention;

FIG. 4 shows on the left side a schematic representation of a test device for generating optical-resolution photoacoustic microscopy images using a water-filled capillary as a waveguide to guide ultrasound from the distal end arranged in the sample to the proximal end of the waveguide, according to one aspect of the present invention;

FIG. 5 shows a simplified schematic diagram according to an embodiment of the present invention with a dual waveguide as the multimode waveguide used to both guide light inside the sample and guide ultrasound sound out of the sample;

FIG. 6 shows a schematic view of a dual waveguide as the multimode waveguide made of a fluid-filled cladding made of silica capillary in which light is guided in the silica part while sound is guided in the inner fluid core according to yet another embodiment of the present invention;

FIG. 7 shows a schematic representation of a dual waveguide made of a fluid-filled hollow optical/acoustical guide, where both light and sound are guided in the fluid core; and

FIG. 8 depicts an alternative embodiment of the dual waveguide shown in FIG. 6, where the propagation in path in water is reduced by inserting a fiber-optic hydrophone into the capillary.

Herein, identical reference numerals are used, where possible, to designate identical elements that are common to the figures. Also, the images in the drawings are simplified for illustration purposes and may not be depicted to scale.

### DETAILED DESCRIPTION

The embodiments of the present invention are directed to an endoscopic system, method, and device that is capable of achieving optical focusing and scanning on a sample, for example tissue, through a multimode waveguide and using the same multimode waveguide to pickup the generated photoacoustic waves, transporting the acoustic signal from the photoacoustic waves to the proximal side of the waveguide where the acoustic signal signal or wave can be detected by a transducer to thereby generate an OR-PAM image at centimeters depth in tissue. The embodiments of the present invention also describe a multimode waveguide that is configured to also pickup and transport optical fluorescence from the sample to the proximal side where the optical fluorescence can be detected to produce a fluorescence image. The embodiments of the present invention further describe a dual modality imaging scheme whereby a photoacoustic image and a fluorescence image are obtained from the sample at the same time by detecting with a transducer that can detect acoustic and fluorescent radiation.

Some of the main features of the embodiments of the present invention include, but are not limited to, a light injection and sound emission for detection that are performed at the same tip, being the proximal tip of the multimode waveguide that is located outside the sample under test, that the light is guided from the proximal tip of the waveguide that is located outside the sample to the distal tip that is located inside the sample. Moreover, sound is guided from the distal tip of the waveguide inside the sample to the proximal tip outside the sample. In addition, the distal tip can be free from any additional components, either optical or acoustical, and the multimode optical waveguide, for example a dual acousto-optic waveguide may have diameters as small as typically a hundred microns. This allows for a smaller area that is needed for insertion of the multimode waveguide into the sample, and allows for minimal invasion.

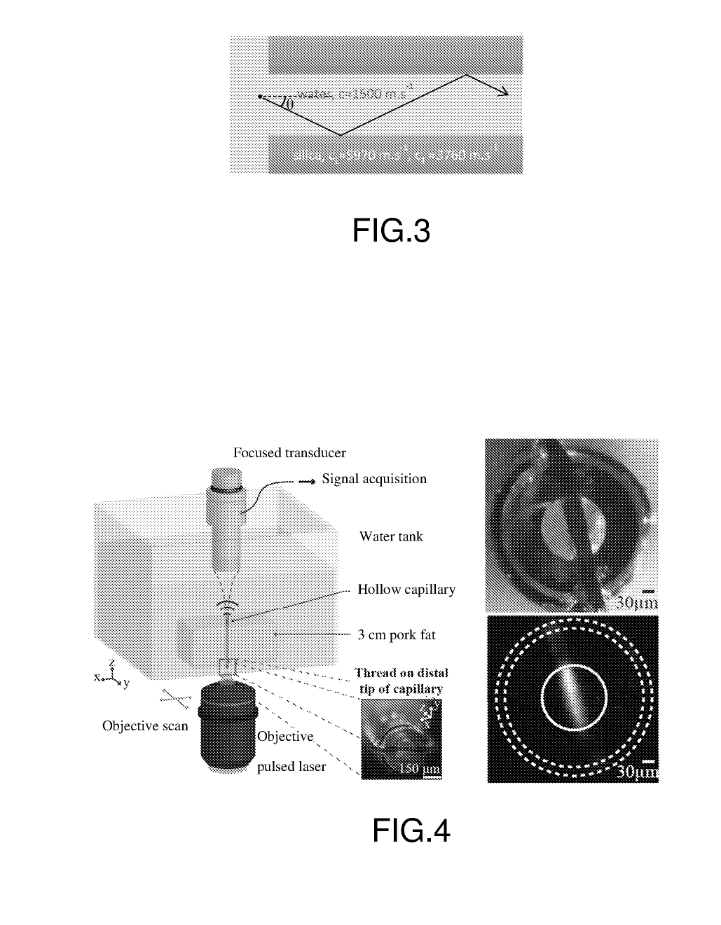

As schematically illustrated in FIG. 3, a multimode waveguide, according to one aspect of the present invention, can exemplary be made as a fiber having a fluid-filled core and a cladding as a silica capillary, in which the sound waves are guided in the fluid-filled core, and the light waves are guided in the cladding. Because of total internal reflection of sound waves at a fluid/silica interface, sound is confined and guided by the liquid core. As a liquid, water can be used.

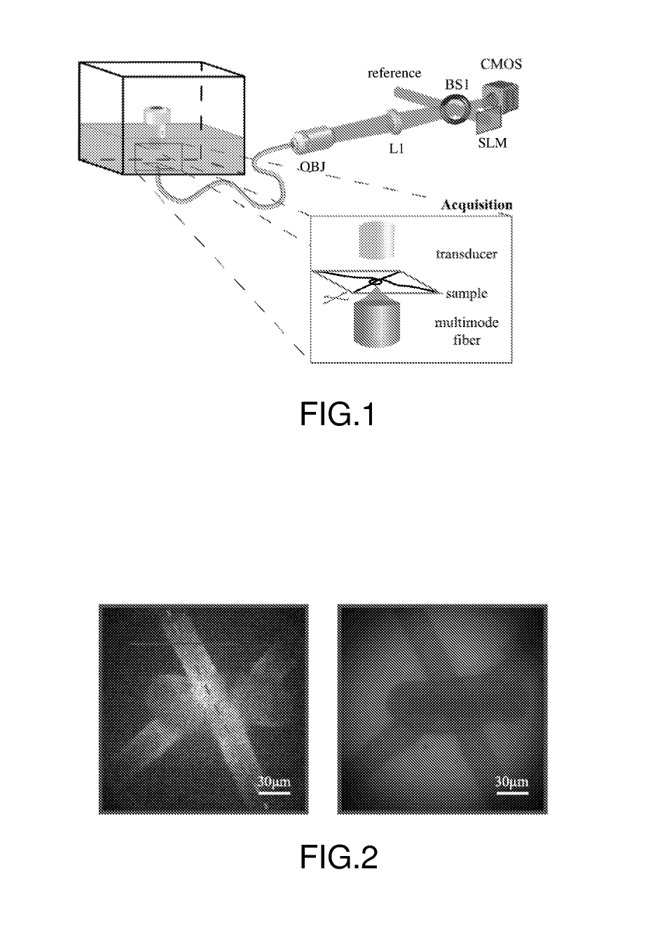

FIG. 4 shows an experimental setup to test the capability of fluid, for example water, as a carrier for the acoustic waves in the multimode waveguide, to guide and transport the sound waves or radiation. For example, the sound can be ultrasound according to another aspect of the present invention. This experimental setup was used to obtain an optical-resolution photoacoustic image through a thick layer of pork by processing the acoustic waves with a processing device. A water-filled capillary as a multimode waveguide is immersed in water, and is embedded in a pork fat layer. As opposed to the setup shown in the background art of FIG. 1, light from a pulsed laser was directly focused by an objective, for example a laser microscope objective, on a sample at the distal tip of a multimode waveguide, and the capillary was used to guide the generated ultrasound wave through the tissue, towards the transducer located at the proximal tip or end of the waveguide located outside the sample. For experimental purposes, the sample is a wire that is attached to the distal end or tip of the waveguide, as shown in the zoomed-in photo depicted in the bottom center of FIG. 4, at the surface of the pork fat layer. After propagating through the water inside the waveguide through the fat layer, and after exiting the proximal tip of the waveguide, the ultrasound is further propagated through the water to reach the transducer, and an optical-resolution photoacoustic image is obtained. This experimental set-up shows the capabilities of fluid as a acoustic wave carrier in the context of optical resolution photoacoustic imaging.

According to one aspect of the embodiments of the present invention, both light excitation and sound detection is combined into a single multimode waveguide, for example a dual-mode acousto-optic waveguide as described above, with both light excitation and sound detection located at the proximal tip that is located outside the sample under test, with a distal tip free of any acoustical or optical components located inside the sample. The distal tip is arranged such that an area of the sample under test is subject to emitted light that causes the photoacoustic effect in the area of the sample. In particular, a dual waveguide can be made of a cladding layer that is filled with fluid serving as the core, for example a fluid-filled silica capillary, with light guided in the cladding, for example the silica shell and sound guided in the fluid core, as further described below.

According to another aspect of the embodiments of the present invention, and innovative concept has been provided by using a dual waveguide to both guide light into the sample and to guide sound outside the sample, in the context of photoacoustic imaging by an endoscopic approach. More specifically, one aspect of the embodiments relies on guiding the light through the sample on the way in via multi-mode optical propagation and detecting ultrasound that was generated at an area of the sample via the photoacoustic effect, after it has propagated on the way out from the sample via mostly mono-mode acoustic propagation. According to another aspect, the multimode waveguide includes a cylindrical elongated component with a small transverse dimension, preferably a few hundred microns in diameter, and having a comparatively large length, preferably from several millimeters to meters. More particularly, the distal tip inserted into the sample under test, for instance biological tissue, is fully passive, which means that it is free of any additional components, such as any electrical conductors, acoustical transducer, optical elements, amplifiers, beam splitters, mirrors, etc., apart from the distal end of the multimode waveguide itself. According to embodiments of the present invention, examples of such multimode waveguide include, but are not limited to, fluid-filled capillaries, fluid-filled optical/acoustical hollow fibers, and fluid-filled needles.

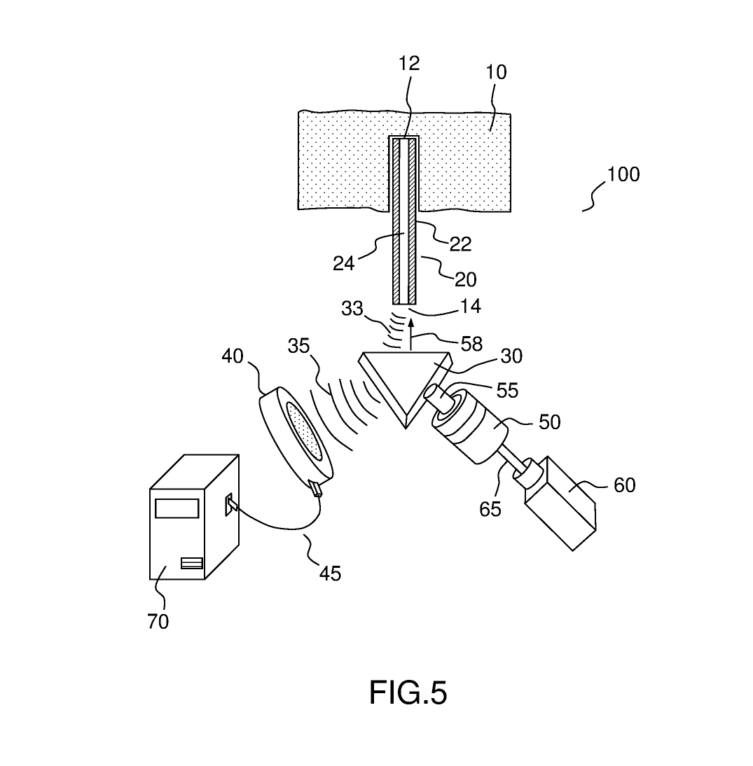

FIG. 5 is a schematic illustration of the method, device, and system 100 of the present invention showing a simplified schematic representation with a dual waveguide 20 having a distal end 12 and a proximal end 14 used to both guide light 58 into the sample 10 and guide ultrasound sound 33 out of the sample 10. The waveguide 20 is introduced into the sample 10 such that distal end 12 is located inside the sample 10, while the proximal end 14 is located outside the sample. Light 65 is generated by an illumination device 60, for example but not limited to a laser, is passed through optics 50 or lenses for focusing light as a focused light beam 55 onto an acousto-optic coupler 30, and the light 58 emitted from the acousto-optic coupler 30 is then introduced into the waveguide 20 via its proximal end 14. The acousto-optic coupler 30 can be implemented as a glass plate or slide that reflects sound radiation or waves, and at the same time is transmissive to light. The glass slide can be, in a variant, be arranged at an oblique angle towards the orientation of the waveguide 20, for example 45°, to deflect ultrasound towards the acoustic transducer 40 while being transparent to laser light. In the variant shown, the acousto-optic coupler 30 is implemented as a prism. Proximal end 14 is located outside the sample 10. The light 58 then propagates inside waveguide 20 along an optical part thereof to reach the distal end 12 of the waveguide 20.

Light 58 then exits at the distal end 12 of the waveguide 20 and impinges on an area of the sample 10, and by a photoacoustic effect of the sample 10. Sound radiation or waves are generated, and at least a portion of the sound generated by the photoacoustic effect propagates back inside the dual waveguide 20 an enters via the distal end 12 and is guided back through waveguide 20 along an acoustic part. The sound is then emitted as sound 33 from the proximal end 14 of waveguide. Sound 33 enters the acousto-optic coupler 30 and is redirected as sound 35 towards an acoustic transducer 40 or sound detection device that can capture the sound 35 and can convert it into data that is transmitted via a data link 45 to a data processing and visualization device 70.

The acousto-optic coupler 30 allows injecting light 58 that originates from light source 60 into waveguide 20 at the proximal end 14, and at the same time can receive the sound 33 from the same location, being the proximal end 14 of waveguide 20, and direct the sound towards a different location where the acoustic transducer 40 is located. Therefore, the proximal end 14 of multimode waveguide 20 has a dual functionality of emitting sound 33 and simultaneously receiving light 58. As a consequence, distal end 12 or distal tip inserted into the sample 10 can be free from any components, for example, it can be free from electrical circuits, ultrasound transducers, conductors, optical elements such as lenses, mirrors, and amplifiers, and this allows to keep the distal tip as small as possible.

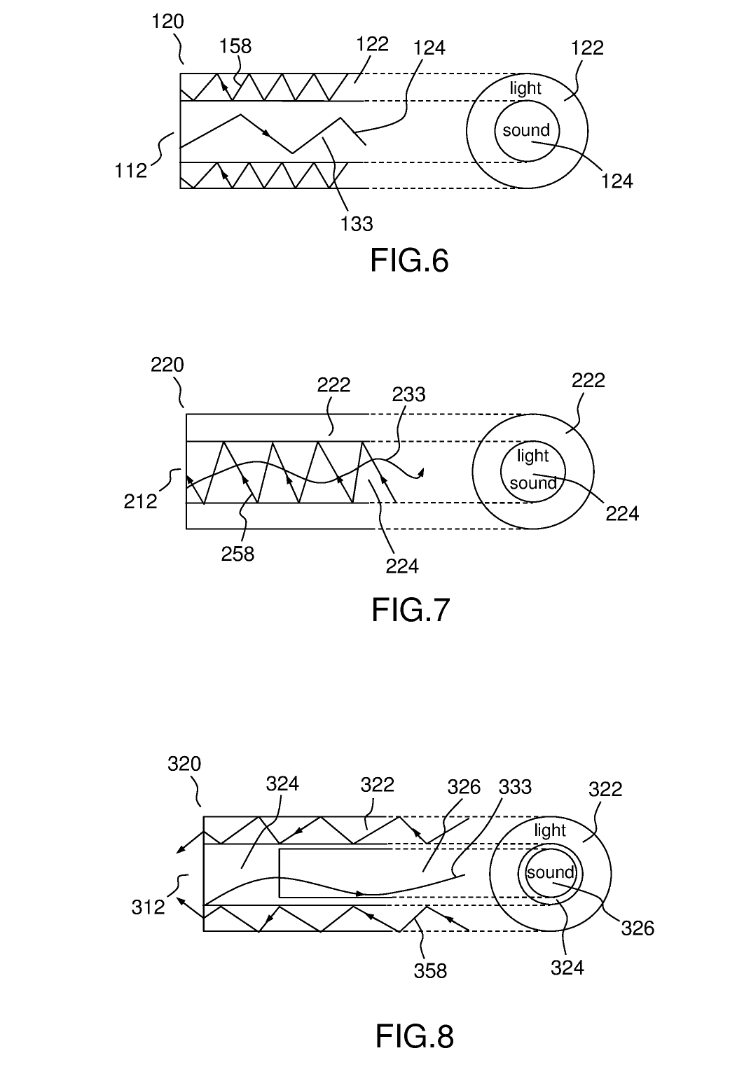

FIGS. 6-8 show schematic representations of different embodiments of multimode waveguides 120, 220, 320 that can be used for the system 100 shown in FIG. 5. For example, FIG. 6 represents a waveguide 120 having a cladding 122, for example a silica cladding layer that has a core 124 filled with fluid. With such waveguide 120 that is used in system 100, light waves or radiation 158 can be guided and confined by the outer cladding 122, serving as a fiber-optic guide, while the sound waves or radiation 133 are propagated and guided through the fluid core 124. The sound waves that were generated by the photoacoustic effect at an area of the sample enter via the distal end 212. In this case, the two waves propagate in two different parts of a multimode waveguide, being a dual waveguide.

FIG. 7 shows a schematic representation of a fluid-filled hollow optical/acoustical waveguide 220. In the variant shown, the cladding 222 can be a conventional hollow-core optical fiber, and the core 224 is filled with fluid. It can also be implemented as metallic needle filled with the hollow core 224 filled with fluid. In this case, both light 258 and sound 233 are guided within the fluid core 224, both reflected by the inner wall of the waveguide 220, as the cladding is made of a material that prevents propagation of light and sound. Therefore, in this variant, the two waves propagate in the same part of the multimode waveguide 220.

FIG. 8 shows a schematic representation of another embodiment of the present invention, showing a waveguide 320. Because acoustic transport of the sound 333 in the fluid core 324 may suffer significant attenuation for very long fiber, waveguide 320 represents an alternative configuration where a fiber optic hydrophone 326, or any fiber-like or needle-like hydrophone, is inserted into the core 324 and is used to reduce the propagation path in the fluid core 324 inside the cladding 322, for example water, by directly detecting the sound 333 at the tip of the hydrophone 326 close to the distal end 312 of the waveguide 320. The propagation in path of the sound 333 in the fluid can be adjusted by varying the insertion length of the hydrophone 326 inside the fluid core 324. Also, in this variant, the light 358 propagates in the cladding 322.

While the invention has been disclosed with reference to certain preferred embodiments, numerous modifications, alterations, and changes to the described embodiments are possible without departing from the sphere and scope of the invention, as defined in the appended claims and their equivalents thereof. Accordingly, it is intended that the invention not be limited to the described embodiments, but that it have the full scope defined by the language of the following claims.

1. An endoscopic device for photoacoustic imaging, comprising:
a multimode waveguide having a distal end and a proximal end;
a light source to provide a light beam to the proximal end of the multimode waveguide;
a transducer to capture acoustic radiation that is emitted from the proximal end of the multimode waveguide; and
a processing device to generate a photoacoustic image based on data from the captured acoustic radiation captured by the transducer,
wherein the distal end of the multimode waveguide is configured to be inserted into a sample, the sample generating the acoustic radiation by a photoacoustic effect.

2. The endoscopic device according to claim 1, further comprising:
an acousto-optic coupler to direct the light beam from the light source to the proximal end of the multimode waveguide and also configured to direct the acoustic radiation received from the proximal end of the multimode waveguide to the transducer.

3. The endoscopic device according to claim 1, wherein the light source includes:
a laser light to generate the light beam, and
a laser optics to focus the light beam generated by the laser light onto the proximal end of the multimode waveguide.

4. The endoscopic device according to claim 1, wherein the multimode waveguide includes:
a fluid core, and
a cladding forming a layer around the fluid core,
wherein the fluid core is configured to guide the acoustic radiation from the distal end to the proximal end, and the cladding is configured to guide light of the light beam from the proximal end to the distal end of the multimode waveguide.

5. The endoscopic device according to claim 1, wherein the multimode waveguide includes:
a fluid core, and
a cladding forming a layer around the fluid core,
wherein the fluid core is configured to guide the acoustic radiation from the distal end to the proximal end, and the fluid core is also configured to guide light of the light beam from the proximal end to the distal end of the multimode waveguide.

6. The endoscopic device according to claim 1, wherein the multimode waveguide includes:
a fluid core arranged at the distal end of the multimode waveguide,
a fiber-optic hydrophone arranged at the proximal end of the multimode waveguide and extending throughout the multimode waveguide but for the distal end, and
a cladding forming a layer around the fluid core and the fiber-optic hydrophone.

7. The endoscopic device according to claim 1, wherein the transducer is also configured to capture fluorescent radiation that is emitted from the proximal end of the multimode waveguide, in addition to the acoustic radiation, the fluorescent radiation generated by the sample.

8. A method to generate a photoacoustic image from a sample with a multimode waveguide, the multimode waveguide penetrating into the sample such that a distal end of the multimode waveguide faces an area of the sample under test inside the sample, the method comprising the steps of:
radiating a proximal end of the multimode waveguide with light from a light source;
guiding the light through the multimode waveguide and guiding sound through the multimode waveguide, the sound being created by the light that exits the distal end of the multimode waveguide and impinges on the area of the sample under test, the area causing a photoacoustic effect generating acoustic radiation that enters the multimode waveguide by the distal end; and
emitting the sound from the proximal end of the multimode waveguide, and capturing the emitted sound by a transducer to generate the photoacoustic image.

9. The method according to claim 8, further comprising the step of:
directing the sound that exits from the proximal end of the multimode waveguide by an acousto-optic coupler towards the transducer, and simultaneously directing the light that exits from the light source towards the proximal end of the multimode waveguide by the acousto-optic coupler.

10. The method according to claim 8, wherein the step of guiding further comprises:
guiding the light in a cladding of the multimode waveguide and simultaneously guiding the sound in a core of the multimode waveguide, the core being a fluid core.

11. The method according to claim 8, wherein the step of guiding further comprises:
guiding the light and simultaneously guiding the sound in a core of the multimode waveguide.

12. An endoscopic system for photoacoustic imaging, comprising:
a sample having an opening;
a dual waveguide having a distal end and a proximal end, the distal end of the dual waveguide arranged inside the opening, an area of the sample facing the distal end of the dual wave guide being under test;
a light source to provide a light beam to the proximal end of the dual waveguide;
a transducer to capture acoustic radiation that is emitted from the proximal end of the dual waveguide; and
a processing device to generate a photoacoustic image based on data from the captured acoustic radiation captured by the transducer,
wherein the acoustic radiation is generated by a photoacoustic effect at the area of the sample, by the acoustic radiation that enters the dual waveguide at the distal end.

13. The endoscopic system according to claim 12, further comprising:
an acousto-optic coupler to direct the light beam from the light source to the proximal end of the dual waveguide and also configured to direct the acoustic radiation received from the proximal end of the dual waveguide to the transducer.

14. The endoscopic system according to claim 12, wherein the light source includes:
a laser light to generate the light beam, and
a laser optics to focus the light beam generated by the laser light onto the proximal end of the dual waveguide.

15. The endoscopic system according to claim 12, wherein the dual waveguide includes:
a fluid core, and
a cladding forming a layer around the fluid core,
wherein the fluid core is configured to guide the acoustic radiation from the distal end to the proximal end, and the cladding is configured to guide light of the light beam from the proximal end to the distal end.

16. The endoscopic system according to claim 12, wherein the dual waveguide includes:
a fluid core, and
a cladding forming a layer around the fluid core,
wherein the fluid core is configured to guide the acoustic radiation from the distal end to the proximal end, and is also configured to guide light of the light beam from the proximal end to the distal end of the dual waveguide.

17. The endoscopic system according to claim 12, wherein the dual waveguide includes:
a fluid core arranged at the distal end of the dual waveguide,
a fiber-optic hydrophone arranged at the proximal end of the dual waveguide and extending throughout the dual waveguide but for the distal end, and
a cladding forming a layer around the fluid core and the fiber-optic hydrophone.

18. The endoscopic system according to claim 12, wherein the transducer is also configured to capture fluorescent radiation that is emitted from the proximal end of the dual waveguide, in addition to the acoustic radiation, the fluorescent radiation generated by the area of sample under test.
